(19) United States
(12) Patent Application Publication
(10) Pub. No.: US 2006/0057207 A1
(43) Pub. Date: Mar. 16, 2006

(54) Fast-disintegrating dosage forms of 5,8,14-triazatetracyclo[10.3.1.02,11.04,9]-hexadeca-2(11),3,5,7,9-pentaene

(75) Inventors: Carl B. Ziegler, East Lyme, CT (US); Barbara A. Johnson, Niantic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/270,766

(22) Filed: Nov. 8, 2005

Related U.S. Application Data
(63) Continuation-in-part of application No. 10/300,608, filed on Nov. 20, 2002.
(60) Provisional application No. 60/334,652, filed on Nov. 30, 2001.
(60) Provisional application No. 60/642,083, filed on Jan. 7, 2005.

(51) Int. Cl. A61K 9/14

(52) U.S. Cl. 424/484

(57) ABSTRACT

A fast disintegrating dosage form of varenicline comprising an effective amount of varenicline or a pharmaceutically acceptable salt thereof and at least one pharmaceutically acceptable excipient, wherein the dosage form disintegrates in a patient's oral cavity in less than three minutes. Also provided are a method for reducing nicotine addiction, aiding in the cessation of, or lessening of tobacco use in a subject by administering to the subject an effective amount of the fast disintegrating dosage form of varenicline or pharmaceutically acceptable salts thereof; a method of treating a disorder or condition by administering an effective amount of the fast disintegrating dosage form of varenicline; and, various methods of manufacturing or forming an immediate dosage form of varenicline.

### CROSS REFERENCE TO RELATED APPLICATIONS

The present application is a continuation-in-part of Ser. No. 10/300,608, filed Nov. 20, 2002, which claims the benefit of Ser. No. 60/334,652, filed Nov. 30, 2001, which is incorporated herein by reference in its entirety.

### 1. FIELD OF THE INVENTION

The present invention relates to pharmaceutical compositions for medicinal uses thereof.

### 2. BACKGROUND ART

Varenicline has the structure:

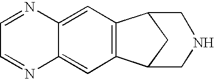

Varenicline is also known as 5,8,14-triazatetracyclo[10.3.1.0^{2,11}.0^{4,9}]-hexadeca-2(11),3,5,7,9-pentaene or 7,8,9,10-tetrahydro-6,10-methano-6H-pyrazino[2,3-h]-[3]-benzazepine. Varenicline and pharmaceutically acceptable acid addition salts thereof are referred to in PCT International Patent Publication No. WO 99/35131, published Jul. 15, 1999, the contents of which are incorporated herein by reference.

Varenicline binds to neuronal nicotinic acetylcholine specific receptor sites and are useful in modulating cholinergic function. Accordingly, this compound is useful in the treatment of various conditions or diseases including, but not limited to, inflammatory bowel disease (including, but not limited to, ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Varenicline is a highly potent compound such that dosage forms are necessarily highly diluted with excipients. The excipients provide dosage forms with adequate stability, while also providing for such desirable features as controlling the drug dissolution (e.g., either fast dissolving or slow dissolving in a controlled-release system as described in U.S. Patent Application Publication No. 2003-0180360 and PCT International Application No. PCT/IB04/01613, the contents of which are hereby incorporated by reference in their entirety), masking bad taste, and providing appropriate properties for preparation of the dosage form (i.e., compression properties for tablets). Finally, varenicline, as a secondary amine, is subject to electrophilic attack. Thus, common excipients such as reducing carbohydrates, can react with the drug.

Because of the high dilution with excipients, reactivity of varenicline with the excipients themselves or with trace impurities (i.e., degradants) of the excipients can be especially problematic. Thus, in the absence of a suitable method for predicting which excipients will provide a balance between stability and other desirable features for dosage formulation, there remains a need for providing appropriate formulations and methods for production of dosage forms of varenicline wherein the drug remains in a predominantly pure form throughout its storage life.

Accordingly, there is a need for providing suitable dosage forms of varenicline, which can be storage-stable for a sufficient time and the dosage can be provided in a relatively pure form over a commercially viable term.

### SUMMARY OF THE INVENTION

The present invention provides a fast disintegrating dosage form of varenicline including an effective amount of varenicline or a pharmaceutically acceptable salt thereof, and at least one pharmaceutically acceptable excipient, wherein the dosage form disintegrates in a patient's oral cavity in less than three minutes. More preferably, the dosage form disintegrates in a patient's oral cavity in less than two minutes and most preferably the dosage form disintegrates in a patient's oral cavity in less than one minute. Further, the present invention provides a method for reducing nicotine addiction, aiding in the cessation of, or lessening of tobacco use in a subject by administering to the subject an effective amount of the fast disintegrating dosage form of varenicline or pharmaceutically acceptable salts thereof. Additionally, the present invention provides a method of treating a disorder or condition by administering an effective amount of the fast disintegrating dosage form of varenicline. Finally, the present invention provides various methods of manufacturing or forming an immediate dosage form of varenicline.

### DETAILED DESCRIPTION OF THE INVENTION

Generally, the present invention relates to novel pharmaceutical dosage forms of varenicline: 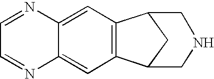
Varenicline is a drug that binds to neuronal nicotinic acetylcholine specific receptor sites, and is useful in modulating cholinergic function. For the present invention, the active ingredient can be used per se or in the form of its pharmaceutically acceptable salt, solvate and/or hydrate. Although any pharmaceutically acceptable form of varenicline can be used in connection with the present invention, it is preferable to use a salt form of the drug. A particularly preferred salt form of the drug is the L-tartrate salt. In the present invention, any solid immediate release dosage form can be used. These include, but are not limited to, immediate release tablets, fast disintegrating dosage forms, chewable dosage forms, and the like. Fast disintegrating dosage forms of the present invention are preferred.

More specifically, the present invention relates to fast disintegrating dosage forms (hereinafter, “FDDF”) and methods to prepare them. These FDDFs of varenicline are suitable for administration to a patient and are stable dosage forms with uniform drug distribution and potency.

The FDDFs of varenicline include varenicline or a pharmaceutically acceptable salt thereof. Further, the present invention specifically provides a dosage form that includes either the L-tartrate or citrate salt of 5,8,14-triazatetra-cyclo[10.3.1.0^{2,11}.0^{4,9}]-hexadeca-2(11),3,5,7,9-pentaene. The FDDF of the present invention can be either in the form of an immediate release, a controlled release or both immediate release followed by controlled dosage form. Additionally, the FDDF of the present invention can include a film coating. The present invention also relates to processes for production of these film-coated dosage forms.

In particular, the present invention provides a method for reducing nicotine addiction or aiding in the cessation or lessening of tobacco use in a subject. The method includes steps of administering to a subject an amount of the FDDF of varenicline that is effective in reducing nicotine addiction or aiding in the cessation or lessening of tobacco use. More specifically, the varenicline is either the L-tartrate or citrate salt of 5,8,14-triazatetra-cyclo[10.3.1.0^{2,11}.0^{4,9}]-hexadeca-2(11 ),3,5,7,9-pentaene.

The present invention can be used to treat disorders or conditions including, but not limited to, inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions; dependencies on, or addictions to, nicotine, tobacco products, alcohol, benzodiazepines, barbiturates, opioids or cocaine; headache, stroke, traumatic brain injury (TBI), obsessive-compulsive disorder (OCD), psychosis, Huntington's Chorea, tardive dyskinesia, hyperkinesia, dyslexia, schizophrenia, multi-infarct dementia, age related cognitive decline, epilepsy, petit mal absence epilepsy, senile dementia of the Alzheimer's type (AD), Parkinson's disease (PD), attention deficit hyperactivity disorder (ADHD), Tourette's Syndrome, and any other similar disorder or condition known to those of skill in the art.

The term “immediate-release form,” “IR form,” or “immediate-release (IR) FDDF” as used herein means a dosage form which when taken orally substantially provides the drug in a form available to be absorbed into the systemic circulation within about one hour or less. The immediate release FDDF of the invention provides a maximum blood concentration of varenicline (Cmax) that is greater than or equal to 80% of that realized for the immediate release, non-FDDF tablet dosage form of varenicline disclosed in U.S. Patent Application Publication No. 2003/0180360A1. Additionally, the immediate release FDDF of the invention provides a maximum area under the curve (AUC) that is greater than or equal to 80% of that realized for the immediate release, non-FDDF tablet dosage form of varenicline. The immediate release FDDF of the invention can further include a glidant, disintegrant, and/or a lubricant. The term also is referred to as “FDDF.”

The term “controlled-release form,” “IR form,” or “controlled-release (CR) FDDF” as used herein means a dosage form which when taken orally substantially provides 50% of the total dose of the drug in a form available to be absorbed into the systemic circulation within about one hour to 15 hours. The controlled release FDDF of the invention can further include a glidant, disintegrant, colorant, flavor and/or a lubricant.

The term “Fast-Disintegrating Dosage Form” or “FDDF” as used herein means dosage forms that disintegrate or dissolve to an extent that the patient believes the solid dosage form to be completely disintegrated or dissolved. That is, the patient can no longer detect any significant lumps or large particles of the original solid dosage form. The term “substantially reducing carbohydrate-free” as used herein means less than approximately 20 wt % of a reducing carbohydrate (including, but not limited to, lactose). Preferably, dosage forms prepared in accordance with the present invention contain less than 10 wt % of a reducing carbohydrate, and more preferably, less than 5 wt %.

The term “buccal delivery” as used herein means a method for drug absorption through the buccal (i.e., inner cheek) tissue.

The term “sublingual delivery” as used herein means delivery of the active compound of the present invention across any tissue under the tongue.

The term “varenicline,” as used herein means the parent drug and all pharmaceutically acceptable salts and prodrugs, thereof. The parent drug of varenicline is set forth in International Patent Publication WO 99/35131, published Jul. 15, 1999, the contents of which are incorporated herein by reference.

The term “mgA” refers to the number of milligrams of active drug based on the free base form of the drug.

The term “pharmaceutically acceptable” means the substance or composition must be compatible chemically, physically, and/or toxicologically, with the other ingredients comprising a formulation, and/or the mammal being treated therewith.

The term “pharmaceutically acceptable salt” means non-toxic acid addition salts derived from inorganic and organic acids. Suitable salt derivatives include, but are not limited to, halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartarates, citrates, camphorates, camphorsulfonates, digluconates, trifluoroacetates, and the like.

The term “active ingredient” means a therapeutically active compound, as well as any prodrugs thereof and pharmaceutically acceptable salts, hydrates, and solvates of the compound and the prodrugs.

The term “appropriate period of time” or “suitable period of time” means the period of time necessary to achieve a desired effect or result. For example, a mixture can be blended until a potency distribution is reached that is within an acceptable qualitative range for a given application or use of the blended mixture.

The term “unit dose,” “unit dosage,” or “unit dosage form” means a physically discrete unit that contains a predetermined quantity of active ingredient calculated to produce a desired therapeutic effect. The unit dose or unit dosage can be in the form of a tablet, capsule, sachet, lozenge, thin consumable film, and the like.

The present invention has numerous embodiments. In any of the embodiments, varenicline or any of its pharmaceutically acceptable salts, solvates and/or hydrates can be used. Procedures for making varenicline are described in U.S. Pat. No. 6,410,550, the contents of which are incorporated herein by reference in their entirety. The resolution of racemic mixtures of varenicline is described in WO01/62736, which is incorporated herein by reference in its entirety.

In any of the embodiments, pharmaceutical compositions of varenicline can be desirably administered in doses ranging from about 0.1 mgA up to about 6 mgA per day (where mgA refers to mg of active drug based on the free base form of the drug), more preferably from about 0.5 to 4 mgA/day, and most preferably from about 1 to 4 mgA per day in single or divided doses. Variatons in such dosages, however, necessarily occur depending upon the weight and condition of the subject being treated. Depending on individual responses, dosage levels below the lower limit of the aforesaid range can be more than adequate, while in other cases still larger doses can be employed without causing any harmful side effects. The final pharmaceutical composition is processed into a unit dosage form (e.g., tablet, or lozenge or thin film) and then packaged for distribution. The processing step varies depending upon the particular unit dosage form. For example, a tablet is generally compressed under pressure into a desired shape. Those of skill in the art are well aware of the procedures used for manufacturing the various unit dosage forms.

In any embodiments of the present invention, the active blend of an immediate release fast disintegrating dosage form generally includes one or more pharmaceutically acceptable excipients, carriers, or diluents. The particular carrier, diluent, or excipient used depends upon the means and purpose for which the active ingredient is being applied. In general, an immediate release fast disintegrating dosage form tablet formulation is made of materials including, but not limited to, excipients, diluents, binders, lubricants, glidants, disintegrants, surfactants, colorants, flavors, sweeteners and mixtures thereof. Acceptable excipients include, but are not limited to, starch, mannitol, sorbitol, xylitol, kaolin, calcium sulfate, inorganic salts (e.g., sodium chloride, sodium bicarbonate), acids such as citric acid, powdered cellulose derivatives, microcrystalline cellulose, silicified microcrystalline cellulose, fast dissolving carbohydrates such as Pharmaburst™, tribasic calcium phosphate, ammonium bicarbonate, calcium sulfate, magnesium carbonate, magnesium oxide, poloxamers which are closely related block copolymers of ethylene oxide and propylene oxide, hydroxypropyl methylcellulose, pullulan, gelatin, carrageenan, gums, combinations thereof, and other similar excipients known to those of skill in the art.

To ensure content uniformity of the blend, a volume mean diameter drug substance particle size of less than or equal to about 60 microns is utilized, more preferably less than or equal to about 40 microns and most preferable less than or equal to about 30 microns.

Preferred diluents are mannitol, sorbitol, xylitol, microcrystalline cellulose (e.g., Avicel® PH200, PH102 or PH101 available from FMC BioPolymer, Philadelphia, Pa.), silicified microcrystalline cellulose (such as Prosolv® available from PenWest Pharmaceuticals Co., Patterson, N.Y.), cellulosic polymers such as hydroxypropyl methylcellulose (HPMC) and hydroxypropyl cellulose (HPC), pullulan and fast dissolving carbohydrates (such as Pharmaburst™ available from SPI Pharma, New Castle, Del.). Finally, examples of glidants include, but are not limited to, silicon dioxide, colloidal silicon dioxide, calcium silicate, magnesium silicate, magnesium trisilicate, talc, starch, combinations thereof, and any other similar glidants known to those of skill in the art.

In any of the embodiments, a binder can be added. Suitable binders include, but are not limited to, substances such as celluloses (e.g., cellulose, methylcellulose, ethylcellulose, hydroxypropyl cellulose and hydroxypropyl methylcellulose), polypropylpyrrolidone, polyvinylprrolidone, gelatin, polyethylene glycol, starch, sugars, trehalose, glucose, sorbitol, natural and synthetic gums (e.g., acacia, alginates, carrageenan, xanthan gum, locust bean gum and gum arabic), waxes, combinations thereof, and any other binder substances known to those of skill in the art.

Further, in any of the embodiments, a lubricant can be used in a tablet formulation to prevent the tablet and punches from sticking in the die. Suitable lubricants include, but are not limited to calcium stearate, glyceryl monostearate, glyceryl behenate, glyceryl palmitostearate, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, combinations thereof, and any other suitable lubricants known to those of skill in the art. A preferred lubricant is magnesium stearate. The magnesium stearate is generally present in an amount from about 0.25 wt % to about 4.0 wt %.

Additionally, in any of the embodiments, disintegrants can also be added to the composition to break up the dosage form and release the compound. Suitable disintegrants include, but are not limited to, sodium starch glycolate, sodium carboxymethyl cellulose, calcium carboxymethyl cellulose, croscarmellose sodium, crospovidone, alginic acid, chitosan, methyl cellulose, microcrystalline cellulose, powdered cellulose, lower alkyl-substituted hydroxypropyl cellulose, polacrilin potassium, starch, pregelatinized starch, sodium alginate, combinations thereof, and any other similar disintegrants known to those of skill in the art. Of these, crospovidone, croscarmellose sodium and sodium starch glycolate are preferred, with croscarmellose sodium being most preferred. The croscarmellose sodium is generally present in an amount from about 0.5 wt % to about 6.0 wt %. Generally, the disintegrant comprises from 1 wt % to 15 wt %, preferably from 1 wt % to 10 wt % of the dosage form.

In any of the embodiments, a flavor or sweetener can be added. Flavors can be chosen from natural and synthetic flavors. Flavors useful in the present invention include, but are not limited to, volatile oils, synthetic flavor oils, flavoring aromatics, oils, liquids, oleoresins or extracts derived from plants, leaves, flowers, fruits, stems, combinations thereof, and other similar flavors known to those of skill in the art. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Further oils include, but are not limited to, citrus oils such as lemon, orange, grape, lime and grapefruit and fruit essences including apple, pear, peach, grape, strawberry, raspberry, cherry, plum, pineapple, apricot, and other fruit flavors. Other useful flavorings include, but are not limited to, vanilla, aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), combinations thereof, and other similar flavorings known to those of skill in the art.

Sweeteners that can be used with any embodiment of the present invention include, but are not limited to, corn syrup, dextrose, invert sugar, fructose, and mixtures thereof; saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol; and the like. Also contemplated are hydrogenated starch hydrolysates and synthetic sweetener 3,6-dihydro-6-methyl-1-1-1,2,3-oxathiazin-4-one-2,2-dioxide, particularly the potassium salt (acesulfame-K) and sodium and calcium salts thereof, and any other sweeteners known to those of skill in the art.

Other ingredients such as colorants and titanium dioxide can also be included.

In one embodiment of the present invention, the present invention provides a fast disintegrating dosage form of varenicline comprising an effective amount of varenicline or a pharmaceutically acceptable salt thereof and at least one pharmaceutically acceptable excipient. The dosage form disintegrates in a patient's oral cavity in less than three minutes. The fast-disintegrating dosage form of varenicline is a solid dosage form that does not require water to aid swallowing. Oral fast-disintegrating products have the potential of improving the overall clinical performance of a drug by reducing the incidence of non-compliance. A patient who is busy or traveling is more likely to take an oral fast-disintegrating dosage form, than a standard tablet requiring water. The phrase “completely disintegrate, dissolve or disperse” used in the context of the present invention, means the FDDF dissolves or disintegrates to an extent that the patient believes the solid dosage form to be completely dissolved or disintegrated. That is, the patient can no longer detect any significant lumps or large particles of the original solid dosage form. Instead, at the point in time when the solid dosage from has completely dissolved or disintegrated in the oral cavity of the patient.

These fast disintegrating dosage forms are typically tablets and thin films that dissolve or disperse rapidly when in contact with saliva. In accordance with the present invention, fast disintegrating dosage forms of varenicline dissolve or disintegrate in the oral cavity following administration in a time range from about two seconds to about three minutes. More preferably, disintegration occurs from about two seconds to two minutes, and most preferably from about two seconds to about one minute. The saliva containing the dissolved or dispersed active ingredient is then swallowed.

The preferred formulations of the present invention contain less than about 20% wt reducing carbohydrates. The presence of reducing carbohydrates is detrimental to the drug stability on storage. Reducing carbohydrates are sugars and their derivatives that contain a free aldehyde or ketone group capable of reaction with the secondary amine of varenicline. Examples of reducing carbohydrates include, but are not limited to, monosaccharides, disaccharides, lactose, glucose, fructose, maltose, and other similar sugars known to those of skill in the art. Further, formulations containing dicalcium phosphate are particularly stable. More specifically, stable formulations are produced with more than about 20 wt % dicalcium phosphate.

The compositions of the present invention can be prepared utilizing numerous technologies including, but not limited to, spray drying, freeze-drying, heat molding and disintegrant-based and/or sugar-based excipient addition, sublimation, and consumable thin film manufacture.

Spray-drying is a widely used process in the pharmaceutical industry and can be used to produce highly porous compositions of varenicline. Spray-dried varenicline, as its free base or its suitable salt derivatives, is combined with excipients including, but not limited to bulking agents such as mannitol, disintegrants such as sodium starch glycolate and croscarmellose sodium, acidic ingredients such as citric acid, glidants and lubricants. This mixture can be blended, granulated, and compressed into tablets as described herein. Examples of such methods for other drugs are set forth in U.S. Pat. Nos. 5,587,180, 5,595,761, 5,635,210, and 5,807,576, each of which is hereby incorporated by reference in its entirety.

The process for spray drying varenicline includes the steps of: (a) dissolving varenicline in a spray solution including a suitable solvent and (b) rapidly evaporating the solvent. Suitable solvents are those that varenicline is soluble including, but not limited to, hydroxylic solvents, non-hydroxylic solvents, water, acetone, tetrahydrofuran, methanol, ethanol, isomeric propanols, combinations thereof, and any other suitable solvents known to those of skill in the art. Alternatively, as varenicline is a low dose drug it could be spray dried with one or more non-volatile excipients as a carrier.

Varenicline should have a solubility of at least 1 wt %, and more preferably at least 5 wt % in the selected solvent. Preferably, the solvent is also volatile with a boiling point of 150° C. or less. In addition, the solvent should have relatively low toxicity and be removed from the varenicline to a level that is acceptable according to the International Committee on Harmonization (ICH) guidelines. Removal of solvent to this level can require a subsequent processing step such as tray-drying or fluid bed drying.

In a preferred method, varenicline is spray dried with one or more excipients as a carrier. The rapid evaporation of spray drying achieves yet another advantage, which is the formation of particles having relatively uniform particle size distribution and shape. The particles formed by rapid evaporation have better flow characteristics and are less likely to become segregated during manufacturing, such as during handling to form tablets. This is particularly important for a drug such as varenicline, since the drug itself has a high potency and therefore is used at a low dose. Reducing segregation during manufacture of the dosage form is important to ensure uniformity of dose in the dosage form. Thus, spray-drying reduces segregation during manufacturing of the dosage form by providing varenicline in a form that is easier to handle. Once the varenicline-containing spray dried composition has been formed, several processing operations can be used to facilitate incorporation of the varenicline into a dosage form. These processing operations include drying, granulation, milling, compression, and the like.

In another embodiment of the present invention, effervescent excipients are incorporated into a varenicline solid dosage form. Once the dosage form is placed in the patient's oral cavity, contact with saliva promotes the acid-base reaction leading to rapid disintegration and release of the active varenicline. The effervescent sensation is not only pleasant to the patient, but also tends to stimulate saliva production, thereby providing additional water to aid in further effervescence and disintegration. Thus, once the dosage form (usually a tablet) is placed in the patient's mouth, it can disintegrate rapidly and completely without any voluntary action by the patient. Even if the patient does not chew the tablet, disintegration proceeds rapidly. Upon disintegration of the tablet, the active varenicline is released and can be swallowed as a slurry or suspension or it can dissolve in the saliva prior to swallowing. The varenicline can be in the form of a powder or it can be in microparticulate form with one or more excipients. The drug in microparticulate form is the preferred form as optionally it aids in taste-masking.

It is also preferred that the microparticles do not release the active varenicline in the oral cavity. Thus the varenicline can be transferred to the patient's stomach for dissolution in the digestive tract and systemic distribution of the pharmaceutical ingredient. In this way, the dosage form provides taste-masking.

The combination of the effervescent disintegration agent and the microparticles containing the varenicline provides an effective dosage form for systemic distribution. The microparticles can be relatively fragile microparticles susceptible to release of the pharmaceutical ingredient upon rupture of the microparticle. The tablet can disintegrate with minimal or no chewing, thus minimizing the problem of microparticle rupture. The effervescent disintegration agent includes any compound that evolves gas. The preferred effervescent agents evolve gas by chemical reactions that take place upon exposure of the effervescent disintegration agent to water and/or to saliva in the mouth. The bubble or gas generating reaction is most often the result of the reaction of a soluble acid source such as citric acid and an alkali metal carbonate or carbonate source such as sodium bicarbonate and sodium carbonate. The reaction of these two general classes of compounds produces carbon dioxide gas upon contact with water included in saliva. The dosage form according to this aspect of the present invention can further include one or more additional excipients including flavors, diluents, colors, binders, fillers, and non-effervescent disintegrants and can be chosen from those herein described.

When varenicline is present in microparticles, the microparticle can be provided as a microcapsule or as a matrix-type microparticle. Microcapsules typically incorporate a discrete mass of the pharmaceutical ingredient surrounded by a discrete, separately observable coating of the protective material. Conversely, in a matrix-type particle, the pharmaceutical ingredient is dissolved or suspended throughout the protective material. Certain microparticles can include attributes of both microcapsules and matrix-type particles. For example, a microparticle can incorporate a core by incorporating the pharmaceutical ingredient in first a protective material and then in a coating of a second protective material, which may be the same as or different from the first protective material surrounding the core. Alternatively, a microparticle can incorporate a core including the pharmaceutical ingredient and a coating incorporating the protective material, wherein the coating itself has some of the pharmaceutical ingredient dispersed within it. These and other known microparticle configurations can be employed.

The microparticles desirably are between about 50 and 800 microns mean outside diameter, and more preferably between about 100 and about 250 microns. Other examples of methodsare set forth in U.S. Pat. Nos. 6,210,711, 5,607,697, 5,178,878, 5,503,846, 4,414,198, 5,298,261, 5,188,825, 5,215,756, 5,298,261, 4,687,662, and 4,946,684, the contents of which are hereby incorporated herein by reference in their entirety.

In another embodiment of the present invention, there is provided a method of freeze-drying. Freeze-drying is a common method for preparing porous structures that dissolve rapidly. One embodiment is a freeze-dried tablet that can be produced by admixing a solvent, granular therapeutic agent, and a carrier material such as gelatin. A natural gum such as xanthan gum is then added to the liquid admixture because it behaves synergistically with gelatin as a flocculating agent to improve the ability of the liquid composition to suspend relatively large particles during the manufacturing process. The liquid composition is then placed into one or more shaped depressions in a tray or mold, and then freeze-dried. Examples of such methods are set forth in U.S. Pat. Nos. 6,680,071, 5,738,875, 6,509,040, 5,631,023, 5,843,347, 5,188,825, 4,946,684, 4,642,903, 4,371,516, 4,305,502 and 5,843,347 and PCT International Patent Publications WO 95/01782A2 and WO 00/44351A1, the contents of which are hereby incorporated herein by reference in their entirety.

In yet another embodiment of the present invention, there is provided a heat molding method. Fast disintegrating dosage forms of varenicline can be prepared by a method that employs a heat-molding process that involves the steps of setting a molten composition of varenicline. One embodiment consists of an agar solution of a non-reducing sugar such as mannitol as a binder. Examples of such methods are set forth in U.S. Pat. Nos. 6,375,982, 6,406,717, 5,466,464, and 5,853,758, the contents of which are hereby incorporated herein by reference in their entirety.

A preferred method of preparing a rapid-melt, semi-solid molded composition of varenicline or its suitable salt derivative includes the following steps:
a) melting at least one binder such as lipid materials, polyethylene glycols (PEG), waxes and other fats such as cocoa butter in an amount from about 0.01% to about 70% by weight with a salivating agent such as xylitol, sorbitan monostearate, or polyoxyethylene sorbitan ester in an amount from about 0.05% to about 15% by weight, to form a mixture;
b) mixing a therapeutically effective amount of varenicline with the mixture to form an active mixture;
c) mixing additional excipients such as silicon dioxide, starches, sorbitol, fructose, magnesium stearate, dicalcium phosphate, xylitol, mannitol, maltitol, isomalt, celluloses and mixtures with the active mixture to form a final mixture; and
d) molding the final mixture into the rapid-melt, semi-solid molded composition.
   The rapid-melt, semi-solid molded compositions can contain at least one binder, a salivating agent, an active material, and a diluent/bulking material.

The combination of excipients allows for fast disintegration of the composition when placed in the mouth of a patient. By pressing the composition on or under the tongue, or between the tongue and cheek of the user, the saliva of the user provides hydration to the composition and allows the composition to disintegrate with little or no chewing. The composition becomes a liquid upon the application of pressure. The semi-solid compositions rapidly disintegrate upon the application of pressure by the tongue of the patient, thus forming a liquid carrier for the active ingredients contained therein. The liquid helps provide the unique characteristics and features of the present inventive compositions.

The rapid-disintegrating, semi-solid compositions of the present invention can include at least one binder. As used herein, “binder” means at least one ingredient useful in keeping the composition in its semi-solid state, and can include, without limitation, a high melting point fat or waxy material such as lipid materials, polyethylene glycols (PEG), waxes, and other fats. Preferably, the semi-solid compositions of the present inventive subject matter include a mixture of binders. The binders useful in the compositions of the present inventive subject matter have a melting point of about 25 to 90° C., and preferably about 37° C. When more than one binder is used in the compositions of the present invention, the melting point of the combination of the binders remain within the range of 25 to 90° C., and preferably about 37° C. The present invention uses mixtures of binders. Among the lipid materials useful as binders in the compositions of the present inventive subject matter are those that are commercially available and commonly used in confectionery and other food products. Such lipid materials include, without limitation, cocoa butter, hydrogenated tallow, hydrogenated vegetable oils, hydrogenated cotton seed oil, palm kernel oil, soybean oil, olive oil, stannol esters, and derivatives and mixtures thereof. Hydrogenated vegetable oils (such as hydrogenated palm kernel oil), cocoa butter, and cocoa butter substitutes are among the preferred useful lipid materials.

The amount of binder present in the rapid-disintegrating, semi-solid molded composition of the present invention is from about 0.01% to about 70% by weight of the final composition. Preferably, the amount of binder is from about 0.01% to about 50% by weight of the composition. More preferably the binder is present from about 15% to about 30% by weight of the composition. The rapid-disintegrating, semi-solid molded composition of the present invention can also contain a salivating agent. As is used herein, “salivating agent” means a material that promotes greater salivation in the user of the compositions of the present inventive subject matter. The salivating agent helps create salivation in the mouth of the patient using the compositions of the present invention. This is an important feature since the compositions of the present invention are intended to be taken by the patient without the aid of water to help in the transporting of the composition to the stomach of the patient. The salivating agent can be, without limitation, an emulsifier or a food acid that initiates salivation in the mouth of the patient. Examples of emulsifiers useful as salivating agents in the compositions of the present invention include, but are not limited to, alkyl aryl sulfonates, alkyl sulfates, sulfonated amides and amines, sulfated and sulfonated esters and ethers, alkyl sulfonates, polyethoxlyated esters, mono- and diglycerides, diacetyl tartaric esters of monoglycerides, polyglycerol esters, sorbitan esters and ethoxylates, lactylated esters, phospholipids such as lecithin, polyoxyethylene sorbitan esters, proplyene glycol esters, sucrose esters, combinations thereof, and other similar emulsifiers known to those of skill in the art. The emulsifier can be either saturated or unsaturated. Examples of food acids useful as salivating agents in the inventive compositions include, without limitation, citric acid, malic acid, food salts such as sodium chloride and salt substitutes, potassium chloride, combinations thereof, and other similar food acids known to those of skill in the art.

The amount of salivating agent present in the rapid-melt, semi-solid molded composition of the present invention is from about 0.05% to about 30% by weight of the final composition. Preferably, the amount of salivating agent is from about 0.3% to 20% by weight of the composition. Keeping the amount of salivating agent present in the inventive composition within these limits for weight percentage is important to enhance the desirable properties of the compositions. More particularly, the low amount of salivating agent present in the compositions aids in the compositions retaining the semi-solid state and the rapidity of melting in the mouth of a patient.

The rapid-disintegrating, semi-solid molded compositions of the present invention can further contain additional excipients to aid in the moisturizing of the composition when chewed. That is, the additional excipients aids in the processability of the compositions. The additional excipients can also serve to reduce the concentration of the active materials and add bulk to the composition. Examples of additional excipients useful in the compositions of the present invention include, but are not limited to, silicon dioxide, sugars, starches, sucrose, sorbitol, fructose, talc, stearic acid, magnesium stearate, dicalcium phosphate, erythitol, xylitol, mannitol, maltitol, isomalt, dextrose, maltose, microcrystalline celluloses, combinations thereof, and other similar additional excipients known to those of skill in the art.

The amount additional excipients present in the semi-solid molded compositions is from about 10% to about 90% by weight of the final composition. Preferably, the amount of additional excipients is from about 35% to about 55% by weight of the final composition.

The use of flavors and sweeteners to mask the taste of the active materials can be used with the compositions of the present invention. Thus, other materials, which can be incorporated into the rapid-melt, semi-solid molded composition of the present inventive subject matter include flavors, colors and sweeteners. A distinct feature of the rapid-melt, semi-solid compositions is that they exhibit excellent taste characteristics. Importantly, it is possible to incorporate high levels of flavors, sweeteners and other taste-masking agents if desired. Flavors can be chosen from natural and synthetic flavors. Flavors useful in the present invention include, but are not limited to, volatile oils, synthetic flavor oils, flavoring aromatics, oils, liquids, oleoresins or extracts derived from plants, leaves, flowers, fruits, stems, combinations thereof, and other similar flavors known to those of skill in the art. Further oils include, but are not limited to, citrus oils such as lemon, orange, grape, lime and grapefruit and fruit essences including apple, pear, peach, grape, strawberry, raspberry, cherry, plum, pineapple, apricot, and other fruit flavors. Other useful flavorings include, but are not limited to, mint, peppermint, cinnamon, wintergreen, vanilla, aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), combinations thereof, and other similar flavorings known to those of skill in the art.

Sweeteners that can be used with any embodiment of the present invention include, but are not limited to, corn syrup, dextrose, invert sugar, fructose, and mixtures thereof; saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol; and the like. Also contemplated are hydrogenated starch hydrolysates and synthetic sweetener 3,6-dihydro-6-methyl-1-1-1,2,3-oxathiazin-4-one-2,2-dioxide, particularly the potassium salt (acesulfame-K) and sodium and calcium salts thereof, and any other sweeteners known to those of skill in the art.

It should be recognized that the composition can be prepared by a variety of methods well-known by those of ordinary skill in the art. Such processes can be used on a batch or continuous process format and involve melting the binders and uniformly blending them for suitable periods of time prior to adding the salivating agent. Once these two components have been blended together, further components can be added either together or sequentially until a uniform mixture is obtained. The resulting mixture is in a semi-solid state that can be poured into a mold, cast into preformed shapes, or stamped into the final products. Other tableting techniques can be used.

In a further embodiment of the present invention, there is provided a disintegrant-based fast disintegrating dosage form. Disintegrants in fast dissolving dosage forms affect the rate of tablet disintegration and therefore the dissolution of varenicline. They are widely used in the development of fast-dissolving tablets. Examples of preferred disintegrants suitable for formulating with varenicline include, but are not limited to, sodium starch glycolate and croscarmellose sodium, starch, microcrystalline cellulose, substituted hydroxypropyl cellulose, crosslinked polyvinylpyrrolidone and modified cellulose. Additional disintegrants and such methods to formulate them with the active drug, varenicline, are set forth in European Patent No. EP 1 194 125; PCT International Patent Publication No. WO 01/03672; U.S. Patent Application Publication No. 2002/0068084A1; and, U.S. Pat. Nos. 5,464,632, 5,178,878, 5,503,846, 6,391,335, 6,200,604, 6,365,182, 5,298,261, 5,215,756, 4,371,516, 4,305,502, and 4,946,684, the contents of which are hereby incorporated herein by reference in their entirety.

Another embodiment of the present invention provides a carbohydrate-based fast dissolving dosage form. Using this technology varenicline formulations include a co-processed carbohydrate system and suitable excipients. These formulations are directly compressible into solid dosage forms that rapidly and completely dissolve and/or disintegrate in the oral cavity, preferably within about sixty seconds. The co-processed carbohydrates, and formulations produced therefrom, include particles having a non-filamentous microstructure. The co-processed carbohydrates and the formulations produced therefore are directly compressible into solid dosage forms. The term “co-processed carbohydrate” means the processing of at least two polyols together to make a single product. For example, mannitol and sorbitol can be co-spray dried by first preparing a single solution of mannitol and sorbitol. Co-processing of carbohydrates includes, co-granulating at least two granular or crystalline polyols such as mannitol and sorbitol, co-granulating at least two spray-dried polyols, or co-granulating a spray-dried polyol and a granular or crystalline polyol. Co-processing also includes, but is not limited to, co-spray drying at least two polyols. Examples and means are set forth in U.S. Pat. No. 6,497,899, the contents of which are hereby incorporated by reference. The term “co-processed carbohydrate system” is construed to include a co-processed carbohydrate plus a disintegrant and a glidant.

In one embodiment, a co-processed carbohydrate system formulation of varenicline according to the present invention includes, but is not limited to, at least two co-processed carbohydrates, one or more disintegrating agents such as crospovidone, sodium croscarmellose, sodium starch glycolate, and combinations thereof, and one or more glidants herein before defined.

Carbohydrates useful in the present invention include, but are not limited to, sorbitol, mannitol, erythritol, maltitol, lactitol, isomalt, and mixtures thereof. Examples and means are set forth in PCT International Patent Application No. WO2003-051338. The contents of which are set forth as reference. The tablets produced by this method described in the reference above preferably have a tablet hardness in the range of about 10 newtons to about 100 newtons and a friability (standard USP test method) in the range of about 0.0 percent to about 5 percent.

A further embodiment of the present invention provides a sugar-based fast-dissolving dosage form. Typically these sugars act as bulking agents and often provide a pleasant mouth feel and taste masking. One or more sugar—based excipients from the following list may be formulated with varenicline to prepare a fast-disintegrating dosage form: amylose, dextrose, fructose, sucrose, erythritol, isomalt, lacitol, maltitol, mannitol, sorbitol, starch hydrolysate, polydextrose, glucose and xylitol. In particular, preferred formulations of varenicline using this technology contain less than about 20 wt % of the reducing sugars including lactose, glucose, fructose, maltose and other similar sugars. Additional references to this technology and such methods to formulate them with an active drug are set forth in U.S. Pat. Nos. 5,576,014, 6,589,554, 6,316,027, 6,277,406, 6,171,607, and 6,165,512, the contents of which are hereby incorporated herein by reference in their entirety.

Methods that convert a mixture of spinnable carrier sugars and other processing aids into candy floss are also useful to prepare fast-dissolving dosage forms of varenicline. The candy floss can be milled and blended with varenicline and other excipients and compressed into fast-dispersing tablets. The method can also be used to produce microspheres instead of floss and has applications in the production of fast-dissolving, chewable and sustained release tablets. Similarly, the methods to formulate them with the active drug, varenicline, are set forth in U.S. Pat. Nos. 4,855,326; 5,587,172; 5,622,719; 5,869,098; 5,866,163; and, PCT International Patent Application Nos. WO 95/34293A1 and WO 95/334290A1, the contents of which are hereby incorporated herein by reference in their entirety. The combination of varenicline with starch or cellulose and one or more water soluble saccharides such as erythritol produces a formulation that displays rapid disintegration as disclosed in Murakami, T Proc. Int. Symp. Controlled Bioact. Matter 1999, 26, 855-6. Other fast-dissolving formulation applications employing varenicline as the active pharmaceutical ingredient with high amylose containing starch or polymeric materials including, but not limited to, gelatin, dextran, and dextrin via a steam extruded process are disclosed in U.S. Pat. No. 6,375,982, which is incorporated herein by reference in its entirety.

Another embodiment of the present invention provides a sublimation method. Varenicline can be formulated using sublimation techniques to prepare highly porous compressed tablets. In this method, inert solid ingredients that volatize readily including, but not limited to, urea, ammonium bicarbonate, ammonium carbonate, hexamethylene tetramine, naphthalene, phthalic anhydride, benzoic acid, camphor, menthol are formulated with other excipients and compressed into a tablet. Volatile materials are then removed via sublimation, which generates a porous structure. For example, highly porous, rapidly dispersible tablets can be prepared by using mannitol as a tablet matrix and camphor as the subliming material. Tablets prepared using sublimation techniques exhibit excellent mechanical strength and a high dissolution rate. Examples of such methods to formulate them with the active drug, varenicline, are set forth in US patents and are herein incorporated by reference in their entirety: U.S. Pat. Nos. 3,885,026; 4,134,943; 5,762,961; 5,720,974; 5,529,789; US Patent Application 2002-0002172A1.

A further embodiment of the present invention provides a thin film delivery method. Thin film (or “wafer”) delivery systems of varenicline include stamp-size medicinal strips that quickly disintegrate and/or dissolve when placed in the oral cavity. Typically, thin film dosage forms are based on hydrophilic polymers with film-forming properties. Fillers are used to modify the texture and to improve handling characteristics. Disintegration and release profiles are modified by the addition of disintegrants, amphiphilic substances or poorly soluble polymers. Additional excipients such as flavors and coloring agents are also incorporated. In one preferred embodiment thin film strips vary in thickness from about 30 microns to about 300 microns and can accommodate up to about 10 mg of drug for fast-disintegrating applications, and more preferably from about 40 microns to about 200 microns and can accommodate up to about 5 mg of drug for fast-dissolve applications. In another embodiment the dosage size of the varenicline dosage filmstrip is between 5 to 20 cm² more preferably the dosage film strip is between 3 to 15 cm² and most preferably the dosage film strip is between 1 to 8 cm². The thin film varenicline dosage form can be single or multi-layered, soft and elastic or hard and stiff, smooth-faced or possess a fibrous texture. More preferably, the thin film varenicline dosage form includes a single layer that is soft, elastic, and smooth faced and has a pleasant mouth feel.

Most thin film dosage forms can be produced by any methods known to those of skill in the art including, but not limited to, film coating, casting technology, extrusion technology, and the like. For example, thin film wafers containing varenicline can be manufactured by first preparing a polymeric solution or suspension from the varenicline and inactive excipients. Next the solution or suspension is evenly coated onto a process foil. A multi-stage drying process then removes solvents and solvent residuals. Such methods are set forth in U.S. Pat. Nos. 6,284,264, 6,177,096, 6,153,222, and 5,948,430, the contents of which are incorporated herein by reference in their entirety. Other methods of producing fast disintegrating dosage forms that are films including varenicline are disclosed below: U.S. Pat. Nos. 6,596,298, 6,552,024, and 6,596,298; and, PCT International Patent Publication Nos. WO 00/18365, WO 01170194, WO 03/011259, and WO 02/43657, the contents of which are herein incorporated by reference in their entirety.

The film-forming agent used in the films according to the present invention includes, but is not limited to, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, locust bean gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A preferred film former is pullulan, in amounts ranging from about 0.01 to about 99 wt %, preferably about 30 to about 80 wt %, more preferably from about 45 to about 70 wt % of the film and even more preferably from about 60 to about 65 wt % of the film.

The film of the invention preferably includes pullulan as a film-forming agent and can further include water, additional film-forming agents, plasticizing agents, additional flavoring agents, saliva stimulating agents, cooling agents, surfactants, stabilizing agents, emulsifying agents, thickening agents, binding agents, coloring agents, sweeteners, fragrances, and the like.

Useful saliva stimulating agents are those disclosed herein above for heat-molding processes and in U.S. Pat. No. 4,820,506, which is incorporated herein by reference in its entirety. Saliva stimulating agents include, but are not limited to, food acids such as citric, lactic, malic, succinic, ascorbic, adipic, fumaric and tartaric acids. Preferred food acids are citric, malic and ascorbic acids. The amount of saliva stimulating agents in the film is from about 0.01 to about 12 wt %, preferably about 1 to about 10 wt %, and even more preferably about 2.5 to about 6 wt %. Preferred plasticizing agents include triacetin in amounts ranging from about 0 to about 20 wt %, preferably about 0 to about 2 wt %. Other suitable plasticizing agents include monoacetin and diacetin.

Preferred cooling agents include monomenthyl succinate, in amounts ranging from about 0.001 to about 2.0 wt %, preferably about 0.2 to about 0.4 wt %. A monomenthyl succinate containing cooling agent is available from Mane, Inc. Other suitable cooling agents include WS3, WS23, Ultracool II and the like. Preferred surfactants include mono and diglycerides of fatty acids and polyoxyethylene sorbitol esters, such as, Atmos® 300 and Polysorbate 80. The surfactant can be added in amounts ranging from about 0.5 to about 15 wt %, preferably about 1 to about 5 wt % of the film. Other suitable surfactants include, but are not limited to, poloxamers (Pluronic®) available from BASF Corporation, Florham Park, N.J.), sodium lauryl sulfate, docusate sodium and the like.

Preferred stabilizing agents include xanthan gum, locust bean gum, and carrageenan, in amounts ranging from about 0 to about 10 wt %, preferably about 0.1 to about 2 wt % of the film. Other suitable stabilizing agents include guar gum and the like. Preferred suspending agents include, but are not limited to, triethanolamine stearate, quaternary ammonium compounds, acacia, gelatin, lecithin, bentonite, Veegum® available from R.T. Vanderbilt, Norwalk, Conn., and the like, in amounts ranging from about 0 to about 5 wt %, preferably about 0.01 to about 0.7 wt % of the film.

Preferred thickening agents include methylcellulose, carboxyl methylcellulose, hydroxymethyl propylcellulose and the like, in amounts ranging from about 0 to about 20 wt %, preferably about 0.01 to about 5 wt %. Preferred binding agents include starch, in amounts ranging from about 0 to about 10 wt %, preferably about 0.01 to about 2 wt % of the film.

Suitable sweeteners that can be included are those well known in the art and have been listed previously. The compositions of this invention can also contain coloring agents or colorants. The coloring agents are used in amounts effective to produce the desired color.

In certain methods for preparing films according to the invention, the film-forming ingredients are mixed and hydrated with water separately from the water-soluble ingredients, which are mixed in aqueous solution separately from the organic ingredients and surfactants. In these methods, the final formulation is preferably produced by mixing the film-forming phase with the aqueous phase, then mixing in the organic phase, which includes surfactants, such as Polysorbate 80 and Atmos® 300. This mass is mixed until emulsified. In other embodiments, the aqueous and film forming phases are combined into a single phase by dissolving the water-soluble ingredients in the water and then adding the gums to hydrate. The organic phase is then added to this single aqueous phase.

The resulting formulation is cast on a suitable substrate and dried to form a film. The film is preferably air-dried or dried under warm air and cut to a desired dimension, packaged and stored. The film can contain from about 0.1% to about 10 wt % moisture, preferably from about 3% to about 8 wt % moisture, even more preferably from about 4 to about 7 wt % moisture.

The film-forming phase can include pullulan and stabilizing agents such as xanthan gum, locust bean gum and carrageenan. These ingredients are mixed and then hydrated in water for about 30 to about 48 hours to form a gel. The water is preferably heated to a temperature of about 25 to about 45° C. to promote hydration. The amount of water is about 40 to 80% of the gel. The resulting hydrated gel is then chilled to a temperature of about 20 to about 30° C. for about 1 to about 48 hours. The water is preferably deionized.

The aqueous phase can include ingredients such as coloring agent(s) and sweetener. The water is preferably deionized and the amount of water used is about 5 to about 80 wt % of the final gel mixture. The films that deliver varenicline can also include a triglyceride. Examples of triglycerides include vegetable oils such as corn oil, sunflower oil, peanut oil, olive oil, canola oil, soybean oil and mixtures thereof. A preferred triglyceride is olive oil. The triglyceride is added to the film in amounts from about 0.1 to about 12 wt %, preferably in a range from about 0.5 to about 9 wt %, of the film. The films that contain varenicline also can include a preservative. The preservative is added in amounts from about 0.001 to about 5 wt %, preferably from about 0.01 to about 1 wt % of the film. Preferred preservatives include sodium benzoate and potassium sorbate.

The varenicline containing films can also include a polyethylene oxide compound. The molecular weight of the polyethylene oxide compound ranges from about 50,000 to about 6,000,000. A preferred polyethylene oxide compound is Polyox® WSR N-10 available from Dow Chemical Company, Midland, Mich. The polyethylene oxide compound is added in amounts from about 0.1 to about 5 wt %, preferably from about 0.2 to about 4.0 wt % of the film.

The varenicline containing films can also include propylene glycol. The propylene glycol is added in amounts from about 1 to about 20 wt %, preferably from about 5 to about 15 wt % of the film. The film can be coated to mask the taste of the active ingredient. The coatings that can be used are known to those skilled in the art. These include polymers such, as Eudragit® E available from Röhm America L.L.C., Piscataway, N.J., cellulosics, such as ethylcellulose, and the like.

Additional ways to mask the taste of the active ingredient as known by those skilled in the art are hereby included. One such way is using an ion exchange resin such as Amberlite® IRP-69, available from Rohm and Haas Co., Philadelphia, Pa., and Dow XYS-40010.00, available from the Dow Chemical Co., Midland, Mich.

A further embodiment provides a method relating to taste masking. It is important that the fast dissolving dosage form of varenicline taste pleasant and have good mouth feel. Flavor masking and processing approaches are the two primary methods for achieving taste masking. Flavor masking includes the addition of flavors, sweeteners, acidic amino acids, lipids, and surfactants to attempt to overwhelm the unpleasant taste. Processing approaches include microencapsulation with various cellulosic derivatives, polylactic acid, polyglycolic acid, polyvinyl alcohol, cellulose acetate phthalate, ethyl cellulose, resins and proteins, gelatinized starch, gums, cyclodextrins, chitosan, liposomes, and removal of bitter contaminants by ion exchange resins. Chemical modification and specific salt preparation of an active ingredient have also been used to reduce the unpleasant taste. Microencapsulation processes can be used to taste mask actives and include both physical and chemical processes. Examples of some of these processes include, but are not limited to, coacervation, spray-drying, spray-chilling, spray-congealing, fluidization, phase separation, and extrusion.

Many taste masking technologies are compatible with fast dissolving dosage forms of varenicline or its suitable salt derivative. Disclosed below are taste masking methods suitable for these dosage forms: U.S. Patent Application Publication Nos. 2003-0170310A1, 2003-0124184, 2002-014701, 2003-0068376A1, 2003-0068378A1, 2003-0118653, 2003-0219479A1, and 2004-0013731; U.S. Pat. Nos. 6,221,392, 5,607,697, 6,596,311, 6,589,556, 6,569,462, 6,451,345, 6,139,865, 5,709,886, 6,465,010, 6,284,270, and 6,552,024; European Patent Nos. EP 1 133 282, 1 007 012, and 1 194 125; WO 2004-000197; and, PCT International Patent Publication Nos. WO 2003-059349, WO 2003-000229; WO 02/02080; WO 02/02081, and WO 01/03672, the contents of which are hereby incorporated herein by reference in their entirety.

Other features and embodiments of the invention will become apparent from the following examples, which are given for illustration of the invention rather than for limiting its intended scope.

### EXAMPLES

### Example 1

### Co-Processed Carbohydrate

Varenicline tartrate, 4 g is blended together with equal amounts, 600 g each, of mannitol and sorbitol. The blend is put through a 14 mesh sieve. This blend is granulated in a fluidized-bed granulator using a 15 w/v % glucose solution as a binding agent. Initially the solution is sprayed at a pressure of 2.5 kg/cm²; it is later reduced to 1.5 kg/cm². When the granulation is dry, other ingredients including a suitable flavor, such as peppermint flavor 10 g, and processing aids such as stearic acid 12 g, magnesium stearate 10 g are added to the granulation using a V-blender. The resultant granulation is compressed on a rotary tablet machine. The 540 mg tablets have average tablet hardnesses on the order of 1.5 kp. The tablets are humidified and heated for 20 minutes in a thermo-hygrostat at 35° C. and 85% RH. The tablets are dried for 15 minutes at 50° C. and 30% RH. The tablet hardness increases to a hardness value of about 9 kp after the heat and humidity treatment.

### Example 2

### Co-Processed Carbohydrate

3 Grams of varenicline tartrate and 324 g mannitol is blended and passed through a sieve (20 mesh). The blend is granulated in a fluidized-bed granulator with 100 g of a trehalose solution (20 w/v %) as a binding agent. After drying 0.5% magnesium stearate is mixed with the granulation. The granulation is compressed on a rotary tablet press at a compression pressure of approximately 0.3 ton to produce 200 mg tablets having an average tablet hardness on the order of 1.5 kp. The tablets are stored under heated humidified conditions of 25° C./80% RH for 12 hours, using a thermo-hygrostat and then they are dried for 2 hours at 30° C./40% RH. After the heat and humidity treatment, the tablets have a hardness of about 3 kp.

### Example 3

### Tablets Prepared From Coated Varenicline Tartrate Microspheres and Floss

Varenicline tartrate microspheres are made by a liquiflash process as described in U.S. Pat. No. 5,683,720 using 85% varenicline tartrate, 7.5% carnauba wax and 7.5% Pluronic® F68. The Pluronic® is milled using a FitzMill® (The Fitzpatrick Co., Elmhurst, Ill.) with a 40 mesh screen. All of the ingredients are blended in a high sheer mixer for approximately 10 minutes. (The blend is then subjected to liquiflash processing at 60 Hz and 37% nominal power using the 5″ V-groove heater head disclosed in U.S. Ser. No. 08/874,215, filed Jun. 13, 1997.). The microspheres are sieved. The fraction passing through a 40 mesh and retained on 120 mesh sieve is coated in a fluid bed coater to a 30% coating level using a 1:1 ethylcellulose:hydroxypropylcellulose coating solution dissolved in an acetone:isopropyl alcohol solvent for taste-masking.

Floss Preparation

A preblend of 78.25% sucrose, 11.0% sorbitol, 10.0% xylitol and 0.75% polysorbate 80 (Tween™ 80, Uniqema, New Castle, Del.) is prepared. The floss preblend is processed using the 5″ crown head disclosed in U.S. Pat. No. 5,854,344, at a temperature of 250° C. and rotational speed of 60 Hz (3600 rpm). The floss collected is chopped in a high shear mixer with 2% lactose (2% w/w of the floss) for 2 minutes at 100 rpm with the choppers turned on. Two-hundred proof ethanol (0.5% based on weight of the floss) is sprayed on the chopped floss and mixed. The floss is then dried at 45° C. for 90 minutes with intermittent mixing. The dried, chopped floss is screened through a 20 mesh screen.

Fast Dissolving Tablet Preparation

Fast dissolving tablets are made using the varenicline tartrate microspheres and the floss previously described in this example using the following formulation:

Tablet Formulation

| | |
|---|---|
| Varenicline taste-masked microspheres | 1.13% |
| Floss | 95.70% |
| Grape flavor | 0.70% |
| Citric acid | 1.50% |
| Acesulfame potassium | 0.20% |
| Silicon dioxide | 0.25% |
| Sodium stearyl fumarate | 0.50% |

The coated varenicline microspheres are blended with the sieved floss for 5 minutes in the high shear mixer, followed by the addition of flavors, sweeteners, and citric acid for another three minutes. The silicon dioxide is added and the mix is blended for another two minutes. The final addition of lubricant sodium stearyl fumarate, is blended for an additional two minutes. The blend is then tabletted on a rotary tablet press using 9 mm flat-faced bevel edge punches to achieve a tablet weight of 255 mg and hardness values between 0.5 lb. to 2.0 lb.

### Example 4

### Porous Matrix Bead

A porous matrix bead is made by spray drying an aqueous solution of mannitol:gelatin (95:5). This matrix bead is blended with varenicline tartrate, binder, sweetener, flow agent, colorant, flavors and lubricant to make a tablet with the following composition.

| | Amount in | |
|---|---|---|
| Ingredient | grams | % of Composition |
| Varenicline tartrate | 1.16 | 1.16 |
| Spray Dried Matrix Bead | 15.3 | 77.3 |
| Polyethylene Glycol3350 | 3.2 | 16.2 |
| Sweeteners | 0.6 | 3.0 |
| Cab-O-Sil ® (Cabot Corp., Tuscola, IL) | 0.14 | 0.7 |
| Coloring Agents | 0.1 | 0.5 |
| Flavors | 0.14 | 0.7 |
| Magnesium Stearate | 0.1 | 0.5 |

The mixture is blended for approximately five minutes without the lubricant. The magnesium stearate is added later and blended for an additional 2 minutes. The blend is compressed using ½ inch round punches to produce a tablet weight of approximately 150 mg. The compressed tablets are sintered at 50° C. for 50 minutes in an oven.

### Example 5

### Spray-Dried Varenicline Tartrate

A porous matrix bead is made by spray drying an aqueous solution of varenicline tartrate:mannitol:gelatin (0.4:97:2.6). This matrix bead is blended with other ingredients to make a tablet with the following composition:

| | | % of |
|---|---|---|
| Ingredient | Amount, g | Composition |
| Matrix bead with varenicline tartrate | 231 | 77 |
| Polyethylene Glycol, PEG-3350 | 30 | 10 |
| Tri-Calcium Phosphate | 36 | 12 |
| Cab-O-Sil ® (Cabot Corp., Tuscola, IL) | 3 | 1 |

The mixture is blended for about 3 minutes. The blend is compressed on a rotary tablet press using ½ inch round flat beveled punches to produce a tablet weight of approximately 570 mg. The compressed tablets are sintered at 90° C. for 10 minutes in an oven.

### Example 6

### Effervescent Dosage Forms

Two fast disintegrating effervescent dosage forms of varenicliine tartrate are shown below. Formulation I has a faster disintegration time than Formulation II. After blending these formulations, the tablets are compressed using ½ inch shallow concave punches.

| | |
|---|---|
| Ingredient | Mg per tablet |

Formulation I. (Shorter Disintegration Time)

| | |
|---|---|
| Varenicline tartrate | 1.7 |
| Mannitol | 119.4 |
| Microcrystalline Cellulose, Silicified | 119.4 |
| Sodium carbonate, anhydrous | 47 |
| Sodium bicarbonate | 105 |
| Citric acid, anhydrous | 75 |
| Polyvinylpyrrolidone, cross-linked | 25 |
| Colloidal silicon dioxide | 2.5 |
| Magnesium stearate | 5 |
| Total Tablet weight | 500 |

Formulation II. (Longer Disintegration Time)

| | |
|---|---|
| Varenicline tartrate | 1.7 |
| Mannitol | 270.8 |
| Sodium carbonate, anhydrous | 40 |
| Sodium bicarbonate | 105 |
| Citric acid, anhydrous | 75 |
| Colloidal silicon dioxide | 2.5 |
| Magnesium stearate | 5 |
| Total Tablet weight | 500 |

### Example 7

### Effervescent Table

| | |
|---|---|
| Coated varenicline tartrate (26.3 wt % potency) | 1.0% |
| Mannitol Powdered | 78.7% |
| Sodium Bicarbonate | 2.3% |
| Citric Acid, Anhydrous | 1.7% |
| Artificial sweetener | 4.6% |
| Crosslinked polyvinyl pyrrolidone | 5.8% |
| Colloidal Silicon dioxide | 0.3% |
| Magnesium Stearate | 1.5% |
| Artificial Flavor | 3.8% |
| Artificial Color | 0.3% |
| Tablet Weight | 650.0 mg |

All of the ingredients shown above are weighed and blended in a V-blender for about 30 minutes. The magnesium stearate is added and the mixture is blended for about an additional 5 minutes. The blend is compressed on a rotary press resulting in 650 mg tablets with average hardness values of 35-50 Newtons.

### Example 8

### Heat Molded Dosage Form

190 Grams of cocoa butter, 3.4 grams of lecithin and 10 grams of sorbitan monostearate are melted. 20 grams of polyethylene glycol and 2.0 grams polyoxyethylene sorbitan ester is added to the melt. The mixture is mixed for 8 minutes at 120° F., then for another 2 minutes at 110° F. Following the mixing period, 751.8 grams of xylitol powder is added to the mixture along with 0.9 grams of aspartame and 0.6 grams of acesulfame K. The mixture is mixed for 5 minutes at 120° F. 13.6 grams of varenicline tartrate is added and the mixture is mixed for 7 minutes. 0.8 grams of colorant, 2.0 grams of vanilla flavoring and 34.4 grams of peppermint is added to the mixture, resulting in 1029.5 grams of mixture which is mixed for an additional 10 minutes until all the ingredients are thoroughly mixed. The final mixture is molded into the final product resulting in 130 mg per molded dosage form.

### Example 9

### Heat Molded Dosage Form

A suspension is made by mixing 2 parts of varenicline tartrate, 195.5 parts of mannitol and 0.5 parts of aspartame to 100 parts of 0.4% agar aqueous solution. A 255 mg portion of the suspension is filled into a mold of 10.5 mm in diameter and dried at 30° C. under vacuum to remove the water. This results in a fast disintegrating solid dosage form.

### Example 10

### Disintegrant-Based Dosage Form

Rapidly disintegrating tablets are made from varenicline tartrate multiparticulates using the following formulation.

| | |
|---|---|
| coated varenicline tartrate (with 10% ethylcellulose) | 1.9 mg |
| microcrystalline cellulose | 272.1 mg |
| pregelatinized starch (Starch 1500 ®, | 188.5 mg |
| Colorcon, West Point, PA) | |
| aspartame | 20.0 mg |
| flavor | 15.0 mg |
| magnesium stearate | 2.5 mg |
| Total | 500 mg |

The varenictine tartrate drug particles are coated in a fluidized bed by spraying a solution of ethylcellulose in an ethanol/acetone mixture. The other excipients except the magnesium stearate are sieved and mixed with the coated varenicline tartrate particles in a tumbling blender under dry conditions. Lastly the magnesium stearate is added and the mixture is blended an additional 5 minutes.

The mixture is compressed on a rotary tablet press using 16 mm standard round concave (a radius of curvature equal to 20 mm) punches and about 165K Newtons of pressure. The tablet hardness is ˜50 Newtons and the tablets disintegrate rapidly in the mouth.

### Example 11

### Sublimation-Based Dosage Form

Porous tablets are made by blending the following ingredients and then compressing the mixture into tablets of 0.5 inch diameter and 0.130 inch thickness. The tablets are heated at 72° C. under vacuum for 18 hours. This temperature is above the melting point of PEG 3350 and removes the ammonium bicarbonate from the tablet resulting in a porous matrix with a tablet hardness of about 4 kp.

| INGREDIENTS | MG/TABLET |
|---|---|
| Ammonium bicarbonate (pore former) | (300.00) later removed |
| Varenicline tartrate | 1.7 |
| Mannitol | 166.30 |
| Banana flavor | 8.00 |
| Aspartame | 4.00 |
| PEG 3350 | 20.00 |
| PVP-XL | 40.00 |
| Sodium stearyl fumarate | 10.00 |
| TOTAL | 250.00 |

### Example 12

### Sublimation-Based Dosage Form

The following ingredients are blended and milled to provide a homogeneous mixture. The mixture is heated to melt the menthol and dissolve the hydroxypropyl cellulose (Klucel® EF, Hercules Inc, Aqualon Div, Wilmington, Del.). The molten suspension is heated to 66° C. and stirred for approximately 30 minutes.

Each 1,000 mg suspension contained the following:

| Ingredients | mg/tablet |
|---|---|
| Menthol | (900) |
| | later removed |
| Klucel ® EF | 39.2 |
| PEG 3350 | 39.1 |
| Aspartame | 10.0 |
| Orange flavor | 10.0 |
| Varenicline | 1.7 |
| Total | (1,000) |
| | molten suspension |
| Total after sublimation | 100.0 |

A die-and-punch assembly is precooled in a dry-ice container (−78° C.). 1,000 mg of molten suspension is charged into the tableting die with the bottom punch in place. Once frozen, the solid tablet (frozen suspension) is removed from the die. The solid tablet is heated at 43° C. under vacuum in a sublimator for 18 hours to remove the menthol. The final tablet is about 90% porous. The resultant tablet has good friability properties and rapidly disintegrates.

### Example 13

### Freeze-Dried Based Dosage Form

(a) Preparation of Varenicline 0.682 wt % Solution

Gelatin (765 g) and mannitol (540 g) are added in a portion of purified water (16 kg) by mixing thoroughly in the bowl of a vacuum mixer. The mix is then heated to 40° C.±2° C. and homogenized for ten minutes to allow complete dissolution of the solids. The mix is cooled down to room temperature (20-24° C.): When cooled, the varenicline tartrate (10.2 g), the aspartame (90 g), and mint flavor (90 g) are added sequentially to the mix. The mix is then homogenized to ensure complete dissolution of the solids. The remaining water (492 g). is added to the mixer and the bulk mix homogenized to ensure dissolution is complete.

(b) Preparation of Varenicline 1 mg Units.

250 Milligrams of varenicline 0.682 wt % solution formed in (a) above is dosed into each of one of a series of pre-formed blister pockets having a pocket diameter of about 12 mm. The blister laminate comprises 200 microns PVC (polyvinyl chloride) coated with 40 gsm PVdC (polyvinyl dichloride). The product is frozen immediately in a liquid nitrogen freeze tunnel. The frozen product is then stored below −20° C. for a minimum of 12 hours prior to freeze drying in a freeze drier using a drying temperature of +10° C. and a chamber pressure of 0.5 mbar. The freeze-dried units are then inspected for the presence of critical defects and the remainder of the batch is sealed with lidding foil consisting of a paper/foil laminate (20 μm aluminum).

### Example 14

### Freeze-Dried Based Dosage Form

(a) Preparation of Varenicline Free Base 0.4 wt % Solution

Gelatin (792 g) and mannitol (594 g) are added to a portion of purified water (16 kg) by mixing thoroughly in the bowl of a vacuum mixer. The mix is then heated to 40° C.±2° C. and homogenized for ten minutes. The mix is cooled down to room temperature (20-24° C.). When cooled the varenicline free base (72 g) is added. The mix is homogenized to ensure dissolution of the drug. Citric acid (166 g) is. added gradually with stirring, to adjust the solution pH to 3.0. The remaining water (436 g) is added to the mixer and the bulk mix homogenized to ensure dissolution is complete.

(b) Preparation of Varenicline 2 mg Units

500 Milligrams of the varenicline 0.4 wt % solution, as formed in (a) above is dosed into each one of a series of pre-formed blister pockets having a pocket diameter of 16 mm. The blister laminate comprised 200 μm PVC coated with 40 g per square meter PVdC. The product is frozen immediately in a liquid nitrogen freeze tunnel. The frozen product is then stored below −20° C. for a minimum of 12 hours prior to freeze-drying in a freeze drier using a drying temperature of +10° C. and a chamber pressure of 0.5 mbar. The freeze-dried units are then inspected for the presence of critical defects and the remainder of the batch sealed with lidding foil consisting of a paper/foil laminate (20 gm aluminum). Each blister is overwrapped in a preformed sachet by placing the blister in the sachet and sealing the open end of the sachet completely.

Each dosage form or unit has the following composition:

| Ingredient | Weight(mg) | % by weight of composition |
|---|---|---|
| Purified water USP/EP* | 455 | 91 |
| Varenicline free base | 2 | 0.4 |
| Gelatin EP/USNF | 22 | 4.4 |
| Mannitol EP/USP | 16.5 | 3.3 |
| Citric Acid EP/USP | 4.6 | 0.9 |

*Signifies removal during the lyophilization process

### Example 15

### Sugar-Based Dosage Forms

A shearform matrix material as defined in U.S. Pat. No. 5,866,163 is prepared in accordance with the formula set forth below: Sugar (Sucrose) 84.75%; Binding Agent (Sorbitol) 15%; Surfactant (Tween™ 80, Uniqema, New Castle, Del.) 0.25%; Total 100%.

The sucrose and sorbitol are mixed until a homogenous blend is produced. To this mixture, the surfactant is added and mixed. The blend is then subjected to flash flow processing in an Econo Floss Machine No. 7025 at approximately 3,600 rpm at a temperature setting of high. The spun material is collected as a floss and is macerated in a mixing machine for about 45 seconds. The resulting material is a reduced volume shearform matrix in uncured condition.

A varenicline tartrate mixture is prepared in accordance with the present invention in accordance with the formula set forth below:

Ingredient By Weight: Shearform Matrix Floss, 92 g, varenicline tartrate, 0.57 g, High Intensity Artificial Flavor, 6 g, Sweetener (Aspartame), 0.8 g, Lecithin (Yelkin DS); 0.35 g, Silica (Syloid® 244, Grace Davison, Cambridgeshire, UK), 0.25 g, Orange Color, 0.05 g; Totals 100 g.

The lecithin and varenicline tartrate are mixed and added to the ground floss material. The ingredients are mixed in a mechanical mixing apparatus for 15-20 seconds. The flavors, high intensity sweetener, and Syloid® are then added and mechanically mixed with an additional 10-15 seconds. Finally, the color is added and mixed until the blend takes on a homogenous orange color. The mixture has a homogenous density and excellent flow characteristics. The mixture is added in portions of 0.3 grams to a die having a 0.3 inch diameter. The ingredients are then tamped at a pressure of 80 psi. The tamped dosage units are then cured. Some of the tablets are cured for one day at room temperature and then the packages are sealed.

### Example 16

### Consumable Film Dosage Form

165.4 Grams of Kollidon® 30 (BASF Corporation, Florham Park, N.J.) are dissolved in a solution of 720 ml water and 2660 ml ethanol at ambient temperature with stirring. 220.5 g hydroxypropyl methylcellulose is then added at 55-60° C. and stirred vigorously until clear and homogeneous. The mixture is then allowed to cool. While stirring 78.75 g flavor is added followed by a mixture of 28.88 g varenicline free base and 31.5 g caramel liquid dissolved in 120 ml water. The clear, tan-colored solution is allowed to cool to room temperature and coated onto a suitable carrier material, for example non-siliconized, polyethylene-coated kraft paper using conventional coating/drying equipment. Coating gap and web speed are adjusted to achieve a dry film thickness between 20 and 50 micrometer. The drying temperature depends on the length of the drying oven and the web speed and is adjusted to remove the solvents completely, or almost completely, from the film. The dry film is cut into pieces of a shape and size suitable for the intended use so as to deliver a varenicline dose between 1-2 mg per piece.

### Example 17

### Consumable Film Dosage Form

Pullulan film composition of varenicline tartrate is prepared using the following steps:
A. dissolve aspartame (1.9 mg), acesulfame potassium salt (0.68 mg) and varenicline tartrate (4 mg) in purified water to form an aqueous mixture;
B. mix pullulan (21.8 mg), xanthan gum (0.08 mg), locust bean gum (0.95 mg) and carrageenan (0.41 mg) together in powder form to form a powder mixture;
C. add the powder mixture from step B to the aqueous mixture from step A to form a hydrated polymer gel;
D. stir the hydrated polymer from step C at slow speed (about 50-100 RPM) overnight at room temperature;
E. mix and dissolve monomenthyl succinate (0.14 mg), olive oil (0.68 mg), and menthol (2.7 mg) and propylene glycol (4.1 mg);
F. add titanium dioxide (0.34 mg), Polysorbate 80 (0.47 mg) and Atmos® 300 (0.47 mg) to the oil mixture from step E;
G. add the oil mixture from step F to the hydrated polymer gel from step D and mix until uniform;
H. cast the uniform mixture from step G on a suitable backing; and,
I. dry the cast mixture to form a film.

Approximate dose weight assuming complete evaporation of water from the film after drying is 38 mg.

### Example 18

### Consumable Film Dosage Form

15 Grams of sorbitol, 6 g of glycerol, 0.5 g of polysorbate 80 (Tween™ 80, Uniqema, New Castle, Del.), 2 g of Brij™ 35 (Uniqema, New Castle, Del.), 25 g of lemon mint flavor, 3 g of aspartame, 15 g of 1-menthol, 8 g of varenicline tartrate and 3 g of citric acid are stirred at 60° C. in a mixture of 250 g water and 250 g ethanol until a clear solution has been formed. To the solution, 30 g of hydroxypropyl methylcellulose are added slowly under stirring until a clear and homogeneous solution has been formed. The resulting solution is allowed to cool to room temperature and coated onto a suitable carrier material, for example non-siliconized, polyethylene-coated kraft paper using conventional coating/drying equipment. Coating gap and web speed are adjusted to achieve a dry film thickness between 20 and 50 μm. The drying temperature depends on the length of the drying oven and the web speed and is adjusted to remove the solvents completely, or almost completely, from the film. The resulting film is peeled off the carrier web and cut into pieces of a shape and size suitable for a 1 -2 mg dose of varenicline free base (approximately equivalent to 1.7- 3.4 mg varenicline tartrate).

### Example 19

### Consumable Film Dosage Form

Varenicline tartrate (1.45 gm) was dissolved in purified water (120 gm). Following this, potassium sorbate (0.098 gm) was added and dissolved in the aqueous solution. In a separate container xanthan gum (0.098 gm), locust bean gum (0.114 gm), carrageenan (0.49 gm) and pullulan (26.12 gm) were mixed. The mixture of gums was slowly added to the aqueous solution of varenicline tartrate with rapid mixing. The resulting mixture was allowed to stir 2-4 hours to allow the gums to hydrate. After this, glycerin (1.63 gm) was added and the resulting solution was thoroughly stirred. A thin film was then cast as in the example above and after drying 80 strips were cut (each approximately 1.33×1.33 inch square). Varenicline tartrate comprised 1.7 mg/dose or 0.98 mg mgA in each 1.33 inch square film strip.

### Example 20

### Pharmaburst™ Tablet

Varenicline tartrate was tableted as described below using Pharmaburst™ from SPI Pharma. Varenicline tartrate (0.171 gm—1.71% of total formulation) was added to Pharmaburst™ B2 (9.68 gm—96.8% total formulation) (SPI Pharma, New Castle, Del.) in an amber glass bottle. The two components were blended using a Turbula® Shaker Mixer (Glen Mills Inc, Clifton, N.J.) for 20 minutes. Magnesium stearate (0.15 gm—1.5% of total formulation) was then added to the blend. This mixture was then blended using a Turbula® Shaker Mixer for an additional 3 minutes. The lubed mixture was tableted as 100 mg/tablet on the Manesty F-Press (Thomas Engineering Inc., Hoffman Estates, Ill.) using ¼″ flat face beveled tooling. The resultant 1 mgA varenicline tablets had a tablet hardness of about 6 kP (range 4-8 kP).

### Example 21

### Pharmaburst™ Tablet

Fast disintegrating tablets are made with coated varenicline tartrate and Pharmaburst™ B2 using the following formulation (given in mg/tablet):

| | |
|---|---|
| coated varenicline tartrate (with 10% ethylcellulose) | 1.9 mg |
| Pharmaburst ™ B2 | 96.6 mg |
| magnesium stearate | 1.5 mg |

Coated varenicline tartrate is added to Pharmaburst™ B2 (SPI Pharma, New Castle, Del.) in an amber glass bottle. The two components are blended using a Turbula® Shaker Mixer (Glen Mills Inc, Clifton, N.J.) for 20 minutes. Magnesium stearate (0.15 gm—1.5% of total formulation) is added to the blend. This mixture is then blended using a Turbula® Shaker Mixer for an additional 3 minutes. The lubed mixture is tableted as 100 mg/tablet on the Manesty F-Press using ¼″ flat face beveled tooling to a tablet hardness of about 6 kP.

### Example 22

### Pharmaburst™ Tablet

Fast disintegrating tablets are made using varenicline tartrate and Pharmaburst™ B2 using the following formulation (given in mg/tablet):

| | | |
|---|---|---|
| varenicline tartrate | 1.7 | mg |
| Pharmaburst ™ B2 | 94 | mg |
| flavor | 2 | mg |
| artificial sweetener | 0.25 | mg |
| sodium stearyl fumerate | 2 | mg |

Varenicline tartrate, Pharmaburst™ B2, flavor and artificial sweetener are blended for 20 minutes in a glass bottle using a Turbula® Shaker Mixer (Glen Mills Inc, Clifton, N.J.). The lubricant sodium stearyl fumerate is added and the mixture is blended an additional 3 minutes in a Turbula® Shaker Mixer mixer. The lubed mixture is tableted as 100 mg/tablet on a Manesty F-Press using ¼″ flat face beveled tooling to obtain a tablet hardness of about 6 kP.

### Example 23

### Pharmaburst™ Tablet Made With Varenicline Multiparticulates

Fast disintegrating tablets are made using microparticulates of varenicline. The multiparticulates are made by a melt spray congeal process using the following formulation.

Varenicline is incorporated into multiparticulates to provide taste masking of the drug using the following procedure. The multiparticulates consist of 35 wt % Varenicline in a carrier of 55 wt % glyceryl mono-, di- and tribehenates (Compritol® 888 ATO from Gattefossé Corporation of Paramus, N.J.) and 10 wt % of poloxamer 407 (commercially available as Pluronic® F127 or Lutrol® F127 from BASF Corporation of Florham Park, N.J.). To form the multiparticulates about 2200 g of the Compritol® and about 400 g of the Pluronic® are added to a sealed, jacketed stainless-steel tank (e.g., a Malto-Mat-Universal MMU 5, Krieger AGG, Switzerland) equipped with counter-rotating mixing paddles and a homogenizer. Heating fluid at about 92° C. is circulated through the jacket of the tank. After about 60 minutes, the mixture is melted. The mixture is then mixed at about 80 rpm for about 60 minutes. Next, about 1400 g of Varenicline is added to the melt and homogenized for about 5 minutes, resulting in a feed suspension of the Varenicline in the molten components.

Using a gear pump, the feed suspension is pumped at a rate of about 140 g/min to the center of a spinning-disk atomizer. The spinning disk atomizer consists of a bowl-shaped stainless steel disk of 10.1 cm (4 inches) in diameter. The surface of the disk is heated with a thin film heater beneath the disk to about 90° C. The disk is rotated at about 3500 to about 6000 rpm while forming the Varenicline multiparticulates. The particles formed by the spinning-disk atomizer are congealed in ambient air to form the multiparticulates.

The varenicline tartrate multiparticulates are incorportated into the following formulation with Pharmaburst™ B2 (given in mg/tablet):

| | | |
|---|---|---|
| varenicline multiparticulate | 2.85 | mg |
| Pharmaburst ™ B2 | 93 | mg |
| flavor | 2 | mg |
| artificial sweetener | 0.25 | mg |
| sodium stearyl fumerate | 2 | mg |

Varenicline tartrate multiparticulates, Pharmaburst™ B2, flavor and artificial sweetener are blended for 20 minutes in a glass bottle using a Turbula® Shaker Mixer (Glen Mills Inc, Clifton, N.J.). The lubricant sodium stearyl fumerate is added and the mixture is blended an additional 3 minutes in a Turbula® Shaker Mixer mixer. The lubed mixture is tableted as 100 mg/tablet on a Manesty F-Press using ¼″ flat face beveled tooling to obtain a tablet hardness of about 6 kP.

### Example 24

### Granulation Preparation

A granulation is produced using the following procedure: Povidone K-30 USP (240.0 gm) is dissolved into distilled water (1,890.0 gm) with agitation. Mannitol powder USP (11,160 gm) and varenicline tartrate (600.0 gm) are placed in the Zanchetta 50-liter granulator/processor (Romaco USA, Pompton Plains, N.J.). After an initial two-minute dry mix of the powders with the chopper on and the propeller adjusted to 200 rpm, the Povidone K-30 solution is slowly sprayed into the mixing powder bed using an air-driven spray system. The granulation end-point is determined visually by the consistency of the resulting material. The material is then discharged onto trays and dried at 80° C. utilizing supplied dry air for a period of six hours to a moisture content of not more than 0.08%. The dried material is then passed through a hammermill (knives forward) equipped with a U.S. #40 (420 micron) screen. The milled material is then screened through a U.S. #200 (75 micron) screen. The material retained on the U.S. #200 (75 micron) screen, about 50 percent, has a mean particle size of 210 microns and is retained for overcoating.

Several batches of the 5.0% varenicline tartrate granulation produced in accordance with above are overcoated to a 15% coating level weight based on weight of finished granule using a 5-liter fluidized bed spray coating unit. The coating solution consists of Povidone K-30 USP (360 gm), ethylcellulose NF (495 gm), and distilled acetylated monoglycerides (45 gm). These solids are dissolved in a mixture of ethanol (1,350 gm) and acetone NF (6,750 gm) with agitation. Five kilograms of varenicline tartrate granulation (5.0% w/w) are placed into the 5-liter chamber of the fluidized bed coating unit. The bed is fluidized with air heated to 38° C. and the coating solution is sprayed into the fluidized powder bed using atomized spray at a rate of approximately 50 gm/min until a coating level of 15% by weight is reached. After the endpoint is reached, the material is allowed to dry in the fluidized bed for an additional ten minutes to drive off any residual solvents. The mean particle size of the overcoated material is about 300 microns.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

What is claimed is:

1. A fast disintegrating dosage form of varenicline, comprising:
an effective amount of varenicline or a pharmaceutically acceptable salt thereof; and,
at least one pharmaceutically acceptable excipient.

2. The fast disintegrating dosage form of varenicline according to claim 1, wherein said dosage form comprises a pharmaceutically acceptable salt of 5,8,14-triazatetra-cyclo[10.3.1.0^{2,11}.0^{4,9}]-hexadeca-2(11),3,5,7,9-pentaene.

3. The fast disintegrating dosage form of varenicline according to claim 1, wherein said dosage form disintegrates in a patient's oral cavity in less than three minutes.

4. The fast disintegrating dosage form of varenicline according to claim 3 wherein said dosage form disintegrates in a patient's oral cavity within from about two seconds to two minutes.

5. The fast disintegrating dosage form of varenicline according to claim 3 wherein said dosage form disintegrates in a patient's oral cavity within from about two seconds to about one minute.

6. The fast disintegrating dosage form of varenicline according to claim 1, wherein at least one pharmaceutically acceptable excipient is selected from the group consisting of at least one binder, a salivating agent, a diluent, a sweetener, a disintegrant, flavoring and a film coating agent.

7. The fast disintegrating dosage form of varenicline according to claim 6, wherein said excipient is from about 70 wt % to about 95 wt % and is selected from the group consisting of mannitol, xylitol, sorbitol, sucrose, trehalose, aspartame, monomenthyl succinate, glycerol, menthol, xanthan gum, locust bean gum, carrageenan, lecithin, microcrystalline cellulose, powdered cellulose, starch, pregelatinized starch, fast dissolving carbohydrates, silica, colloidal silica, potassium sorbate, acesulfame potassium salt, sodium bicarbonate, calcium carbonate, calcium phosphate dibasic, tribasic calcium phosphate, calcium sulfate, magnesium carbonate, magnesium oxide, poloxamers, hydroxypropyl methylcellulose, citric acid, povidove, pullulan, Brij™35, gelatin, polyethylene glycol, glyceryl mono, di- and tribehenates, sorbitan monostearate, polysorbate 80, cocoa butter, carnauba wax and combinations thereof.

8. The fast disintegrating dosage form of varenicline according to claim 6 wherein said excipient is from about 70 wt % to about 95 wt % and is selected from the group consisting of mannitol, sorbitol, xylitol, microcrystalline cellulose, silicified microcrystalline cellulose, cellulosic polymers, hydroxypropyl methylcellulose (HPMC) and hydroxypropyl cellulose (HPC), pullulan and fast dissolving carbohydrates.

9. The fast disintegrating dosage form of varenicline according to claim 1, further comprising a film coating.

10. The fast disintegrating dosage form of varenicline according to claim 1, wherein said pharmaceutically acceptable salt is selected from the group consisting of L-tartrate salt and a citrate salt.

11. The fast disintegrating dosage form of varenicline according to claim 1, further comprising an effervescent disintegration agent.

12. The fast disintegrating dosage form of varenicline according to claim 11, wherein the effervescent disintegration agent is selected from mixtures of a soluble acid source and an alkali metal carbonate or a carbonate source.

13. The fast disintegrating dosage form of the varenicline dosage form according to claim 1, wherein the form of varenicline is a structure selected from the group consisting of a tablet, floss, microsphere, multiparticulate, capsule, thin-film strip, pill, fast-dissolving tablet, porous matrix bead, effervescent dosage, molded form, coated tablet, and consumable film.

14. The fast disintegrating dosage form of varenicline according to claim 1, wherein said dosage form is a consumable film, formed by (a) dissolving varenicline or a pharmaceutically acceptable salt thereof in a suitable solvent to form a solution; (b) dissolving potassium sorbate in the solution; (c) adding to the solution of a natural or synthetic gum, carrageenan and pullulan in suitable proportion to form a mixture; (d) stirring the mixture so as to allow the gums to hydrate; (e) adding glycerin to the mixture with stirring; and, (f) casting a thin film, thereby to form a consumable film.

15. The fast disintegrating dosage form of varenicline according to claim 1, wherein said dosage form is a tablet, formed by (a) mixing varenicline or a pharmaceutically acceptable salt thereof with a fast-dissolving carbohydrate to form a mixture; (b) blending the mixture to form a blend; (c) adding a lubricant to the blend and further blending the mixture; and, (d) forming tablets from the mixture.

16. The fast disintegrating dosage form of varenicline according to claim 1 wherein the varenicline is taste-masked.

17. The fast disintegrating dosage form of varenicline according to claim 16, wherein the taste-masking agent is selected from the group consisting of cyclodextrin, glyceryl mono-, di- and tribehenates, poloxamers and natural and artificial flavors, and optionally microspheres or coated microspheres or coated drug particles.

18. The fast disintegrating dosage form of varenicline according to claim 1, further comprising an immediate release dosage form suitable for administration to a subject, which dosage form, when dosed to said subject, results in a maximum plasma concentration (Cₘₐₓ) of said varenicline in an initial administration to said subject, which is, on average, greater than 80% of the corresponding Cₘₐₓ determined for an equal dose of said varenicline in the form of an immediate release bolus.

19. A method of treating a disorder or condition selected from the group consisting of inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions; dependencies on, or addictions to, nicotine, tobacco products, alcohol, benzodiazepines, barbiturates, opioids or cocaine; headache, stroke, traumatic brain injury (TBI), obsessive-compulsive disorder (OCD), psychosis, Huntington's Chorea, tardive dyskinesia, hyperkinesia, dyslexia, schizophrenia, multi-infarct dementia, age related cognitive decline, epilepsy, petit mal absence epilepsy, senile dementia of the Alzheimer's type (AD), Parkinson's disease (PD), attention deficit hyperactivity disorder (ADHD), and Tourette's Syndrome, in a subject suffering therefrom, comprising administering to the subject an effective amount of the fast disintegrating dosage form of varenicline of claim 1.

20. A method for reducing nicotine addiction, aiding in the cessation of, or lessening of tobacco use in a subject comprising administering to the subject an effective amount of the fast disintegrating dosage form of varenicline of claim 1.

21. The method according to claim 19, wherein said administering step is further defined as administering the pharmaceutically acceptable salt selected from the group consisting of the L-tartrate and the citrate salt.

22. A method of forming an immediate-release dosage form of varenicline of claim 1 comprising dissolving varenicline in a suitable solvent so as to form a solution suitable to form a spray, and rapidly evaporating the solvent spray.

23. A method of forming an immediate-release dosage form of varenicline of claim 1 by freeze drying comprising the steps of:
admixing a solvent, varenicline, and a carrier material to form a carrier mixture:
adding a natural gum to the carrier mixture to form a dosage form;
placing the dosage form into at least one shaped depressions in a mold;
freezing the contents of the molded dosage form; and,
freeze-drying the dosage form.

24. A method of forming an immediate-release dosage form of varenicline of claim 1 by heat molding comprising the steps of:
melting at least one binder in an amount from about 0.01% to about 70% by weight with an excipient chosen from a salivating agent, a sweetener, and a flavoring agent in an amount from about 0.05% to about 15% by weight to form a mixture;
mixing a therapeutically effective amount of varenicline with the mixture to form an active mixture;
mixing a diluent material with the active mixture to form a final mixture; and,
molding said final mixture into said rapid-melt, semi-solid molded composition; and, cooling the molded composition.

25. A method of forming an immediate-release dosage form of varenicline of claim 1 by sublimation comprising the steps of:
mixing at least one inert solid ingredient with at least one excipient to form a mixture;
compressing the mixture into a tablet; and,
removing volatile materials by sublimation so as to generate a porous structure.

26. A method of forming an immediate-release dosage form of varenicline of claim 1 by melt spray congeal process comprising the steps of:
mixing the at least one pharmaceutically acceptable excipient to form a mixture;
melting the mixture with continued mixing;
adding a therapeutically effective amount of varenicline to the melt to form a suspension;
pumping the suspension using a gear pump to the center of a spinning-disk atomizer;
rotating the disk at a speed of about 3000 to about 6000 rpm so as to form multiparticulates of a pre-selected desired size; and,
congealing the multiparticulates so as to form the immediate-release dosage form.
